(19) United States
(12) Patent Application Publication
(10) Pub. No.: US 2024/0201067 A1
(43) Pub. Date: Jun. 20, 2024

(54) Spectrum Data Acquisition Method, Cell Sorting Method, And Raman Spectroscopy System

(71) Applicant: National Institute of Advanced Industrial Science and Technology, Tokyo (JP)

(72) Inventors: Yuka Akagi, Tsukuba-shi, Ibaraki (JP); Yasuyuki Kida, Tsukuba-shi, Ibaraki (JP); Nobuhito Mori, Tsukuba-shi, Ibaraki (JP); Yuzo Takayama, Tsukuba-shi, Ibaraki (JP)

(21) Appl. No.: 18/555,186

(22) PCT Filed: Mar. 22, 2022
(86) PCT No.: PCT/JP2022/013239

§ 371 Date: Oct. 12, 2023

(30) Foreign Application Priority Data
Apr. 15, 2021 (JP) 2021-068916

(51) Int. Cl. G01N 15/1434; G01N 15/01

(52) U.S. Cl. CPC G01N 15/1434; G01N 15/01

(57) ABSTRACT

Provided is a spectral data acquisition method which can perform laser irradiation more uniformly while further reducing cell damage. The spectral data acquisition method involves receiving, by means of a spectroscope, Raman scattered light output from a cell by spirally irradiating an area in which the cell is present with laser light from the laser light source, and detecting a Raman spectrum in response to the received Raman scattered light by means of a charge-coupled device (CCD) detector.

### TECHNICAL FIELD

The present invention relates to a spectrum data obtaining method, a cell classification method, and a Raman spectroscopy system.

### BACKGROUND ART

There are microscope systems which perform evaluation of cells by using Raman scattered light. Raman scattered light obtainable from a cell indicates distribution, quantities, and so on of molecules, such as those of protein, lipid, and so on, in a cell. Thus, it is possible to discriminate, in a noninvasive and/or unstaining manner, the state of the cell based on a Raman spectrum obtainable from Raman scattered light.

For example, Non-patent Literature 1 discloses a technique for obtaining Raman scattered light, by designating a square region in a cell based on an image obtained by using a quantitative phase microscope (QPM: quantitative phase microscopy) and irradiating it with laser light by using a random scan system. Further, Non-patent Literature 2 discloses a technique for selecting a cell, by measuring the whole region of the cell by performing line scanning to thereby create a Raman image, and using a spectrum of a cell region extracted from the Raman image.

### CITATION LIST

### Non Patent Literature

NPL 1: Nicolas Pavillon, Nicholas I. Smith, “Maximizing throughput in label-free microspectroscopy with hybrid Raman imaging,” Journal of Biomedical Optics, 2015, 20, 016007, DOI: 10.1117/1.JBO.20.1.016007.
NPL 2: Taro Ichimura and six other authors, “Non-label immune cell state prediction using Raman spectroscopy,” Scientific Reports 2016, 6, 37562; DOI: 10.1038/srep37562.

### SUMMARY OF INVENTION

### Technical Problem

On the other hand, in the case of a line irradiation method such as that disclosed in Non-patent Literature 2, selecting of a cell using a Raman image requires time for measurement and/or analysis, and also increases damage to the cell. Raman scattered light originated in a cell is very weak to be sensed sufficiently, so that it may become easier to be sensed if expose time to the cell is extended; however, in such a case, there are problems that damage to the cell becomes larger and the noise becomes higher. Also, in a random scan method such as that disclosed in Non-patent Literature 1, it is difficult to perform laser irradiation uniformly in a specific region in a cell.

The present invention has been achieved in view of the above matters.

### Solution to Problem

For solving the above problems, a mode of the present invention comprises a spectrum data obtaining method comprising steps for: receiving Raman scattered light that is outputted from a cell as a result that laser light is emitted in a turning manner to a region where the cell exists; and detecting a Raman spectrum corresponding to the received Raman scattered light.

Further, the other mode of the present invention comprises a cell classification method comprising a step of classifying a cell based on spectrum data obtained by performing the above method.

Further, a different mode of the present invention comprises a Raman spectroscopy system comprising: a laser source for outputting laser light; a biaxial Galvano mirror for reflecting the laser light in such a manner that a region where a cell exists is to be irradiated by the laser light in a turning manner; a spectrometer for receiving Raman scattered light, that is outputted from the cell; and a CCD (charge-coupled device) detector for detecting a Raman spectrum corresponding to the received Raman scattered light.

### Advantageous Effects of Invention

It becomes possible to emit, in a turning manner or in accordance with an arbitrarily determined pattern, laser light to an arbitrarily selected region in a single cell, and, accordingly, it becomes possible to obtain Raman scattered light from the whole irradiated region. Thus, when obtaining Raman scattered light, the Raman scattered light can be obtained from an arbitrarily selected region in a single cell in a short period of time, while suppressing damage to the cell.

Further, by expanding a region for measurement, Raman scattered light from plural cells can be obtained at the same time.

Further, in the case that the obtained Raman scattered light is treated as that in the form of spectrum data, and multivariate analysis and/or machine learning is to be performed, it becomes possible to classify the kinds and/or activated states of cells. Thus, discrimination of cells can be performed in a noninvasive and/or unstaining manner, and in a short period of time.

### BRIEF DESCRIPTION OF DRAWINGS

FIG. 1 is a figure which explains examples of optical paths in a Raman spectroscopy system according to an embodiment of the present invention.

FIG. 2 is a figure showing an example of a construction of a Raman spectroscopy system according to an embodiment of the present invention.

FIG. 3 is a figure showing examples of scenes wherein approximately circular specific regions in cells are irradiated by laser light.

FIG. 4 is a figure showing examples of spectra of solid palmitic acid.

FIG. 5 is a figure showing examples of obtained Raman spectra.

FIG. 6 is a figure which explains examples of Raman measurement performed by using a point irradiation method and an irradiation method according to an embodiment of the present invention.

FIG. 7 is a figure showing an example of a graph showing obtained Raman spectra.

FIG. 8 is a figure which explains an example of comparison between an obtained Raman spectrum and a spectrum of palmitic acid.

FIG. 9 is a figure which explains an example of comparison between an obtained Raman spectrum and a spectrum of palmitic acid.

FIG. 10 is a figure which explains an example of comparison between an obtained Raman spectrum and a spectrum of palmitic acid.

FIG. 11 is a figure which explains an example of classification of cell types that was performed with respect to cells in floating states by using an irradiation method according to an embodiment of the present invention.

FIG. 12 is a figure which explains an example of classification of cell types that was performed with respect to cells in floating states by using an irradiation method according to an embodiment of the present invention.

FIG. 13 is a figure which explains an example of classification of cell types that was performed with respect to cells in floating states by using an irradiation method according to an embodiment of the present invention.

FIG. 14 is a figure which explains an example of classification of cell types that was performed with respect to cells in floating states by using an irradiation method according to an embodiment of the present invention.

FIG. 15 is a figure which explains an example of classification of cell types that was performed with respect to cells in floating states by using an irradiation method according to an embodiment of the present invention.

FIG. 16 is a figure which explains an example of classification of cell types that was performed with respect to cells in floating states by using an irradiation method according to an embodiment of the present invention.

FIG. 17 is a figure which explains an example of classification of cell types that was performed with respect to cells in adhered states by using an irradiation method according to an embodiment of the present invention.

FIG. 18 is a figure which explains an example of classification of cell types that was performed with respect to cells in adhered states by using an irradiation method according to an embodiment of the present invention.

FIG. 19 is a figure which explains an example of classification of cell types that was performed with respect to cells in adhered states by using an irradiation method according to an embodiment of the present invention.

FIG. 20 is a figure which explains an example of classification of cell types that was performed with respect to cells in adhered states by using an irradiation method according to an embodiment of the present invention.

FIG. 21 is a figure which explains an example of classification of cell types that was performed with respect to cells in adhered states by using an irradiation method according to an embodiment of the present invention.

FIG. 22 is a figure which explains an example of classification of cell types that was performed with respect to cells in adhered states by using an irradiation method according to an embodiment of the present invention.

FIG. 23 is a figure which explains an example of classification that was performed with respect same cells in different states by using an irradiation method according to an embodiment of the present invention.

FIG. 24 is a figure which explains an example of classification that was performed with respect same cells in different states by using an irradiation method according to an embodiment of the present invention.

FIG. 25 is a figure which explains an example of classification that was performed with respect same cells in different states by using an irradiation method according to an embodiment of the present invention.

FIG. 26 is a figure which explains an example of classification that was performed with respect same cells in different states by using an irradiation method according to an embodiment of the present invention.

FIG. 27 is a figure which explains an example of classification that was performed with respect same cells in different states by using an irradiation method according to an embodiment of the present invention.

FIG. 28 is a figure which explains an example of classification that was performed with respect same cells in different states by using an irradiation method according to an embodiment of the present invention.

FIG. 29 is a figure which explains an example of classification that was performed with respect same cells in different states (stimulated with PMA and A23187) by using an irradiation method according to an embodiment of the present invention.

FIG. 30 is a figure which explains an example of classification that was performed with respect same cells in different states (stimulated with PMA and A23187) by using an irradiation method according to an embodiment of the present invention.

FIG. 31 is a figure which explains an example of classification that was performed with respect same cells in different states (stimulated with PMA and A23187) by using an irradiation method according to an embodiment of the present invention.

FIG. 32 is a figure which explains an example of classification that was performed with respect same cells in different states (stimulated with PMA) by using an irradiation method according to an embodiment of the present invention.

FIG. 33 is a figure which explains an example of classification that was performed with respect same cells in different states (stimulated with PMA) by using an irradiation method according to an embodiment of the present invention.

FIG. 34 is a figure which explains an example of classification that was performed with respect same cells in different states (stimulated with PMA) by using an irradiation method according to an embodiment of the present invention.

FIG. 35 is a figure which explains an example of classification that was performed with respect same cells in different states (stimulated with PHA) by using an irradiation method according to an embodiment of the present invention.

FIG. 36 is a figure which explains an example of classification that was performed with respect same cells in different states (stimulated with PHA) by using an irradiation method according to an embodiment of the present invention.

FIG. 37 is a figure which explains an example of classification that was performed with respect same cells in different states (stimulated with PHA) by using an irradiation method according to an embodiment of the present invention.

FIG. 38 is a figure which explains an example of inference of states of Jurkat cells using machine learning and an irradiation method according to an embodiment of the present invention.

FIG. 39 is a figure which explains an example of inference of states of Jurkat cells using machine learning and an irradiation method according to an embodiment of the present invention.

FIG. 40 is a figure which explains an example of measurement that was performed with respect to plural cells by using an irradiation method according to an embodiment of the present invention.

FIG. 41 is a figure which explains an example of measurement that was performed with respect to plural cells by using an irradiation method according to an embodiment of the present invention.

FIG. 42 is a figure which explains an example of measurement that was performed with respect to plural cells by using an irradiation method according to an embodiment of the present invention.

FIG. 43 is a figure which explains an example of measurement that was performed with respect to plural cells by using an irradiation method according to an embodiment of the present invention.

FIG. 44 is a figure which explains an example of measurement that was performed with respect to plural cells by using an irradiation method according to an embodiment of the present invention.

FIG. 45 is a figure which explains an example of measurement that was performed with respect to plural cells by using an irradiation method according to an embodiment of the present invention.

FIG. 46 is a figure which explains an example of measurement that was performed with respect to plural cells by using an irradiation method according to an embodiment of the present invention.

FIG. 47 is a figure which explains an example of measurement that was performed with respect to plural cells by using an irradiation method according to an embodiment of the present invention.

FIG. 48 is a figure which explains an example of measurement that was performed with respect to plural cells by using an irradiation method according to an embodiment of the present invention.

FIG. 49 is a figure which explains an example of measurement that was performed with respect to plural cells by using an irradiation method according to an embodiment of the present invention.

FIG. 50 is a figure which explains an example of measurement that was performed with respect to plural cells by using an irradiation method according to an embodiment of the present invention.

FIG. 51 is a figure which explains an example of measurement that was performed with respect to plural cells by using an irradiation method according to an embodiment of the present invention.

FIG. 52 is a figure which explains an example of measurement that was performed with respect to plural cells by using an irradiation method according to an embodiment of the present invention.

### DESCRIPTION OF EMBODIMENTS

In the following description, embodiments of the present invention will be explained with reference to the figures.

### (Optical Paths in Raman Spectroscopy System)

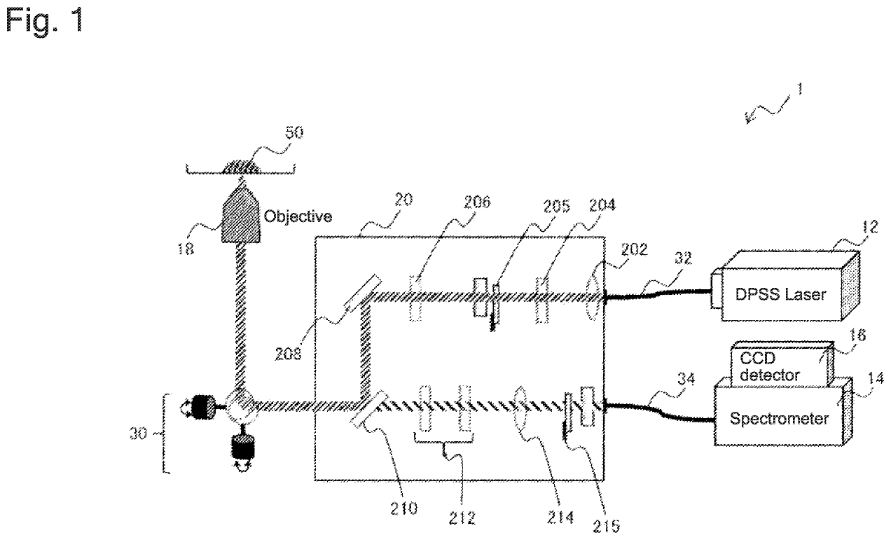

FIG. 1 is a figure which explains examples of optical paths in a Raman spectroscopy system according to a present embodiment. A Raman spectroscopy system 1 according to the present embodiment is a system which can obtain a spectrum from a wide region in a cell at high speed for avoiding changing of a measuring region due to a complicated structure of the cell. The Raman spectroscopy system 1 according to the present embodiment comprises a diode pumped solid state (DPSS) laser light source 12, a spectrometer 14, a CCD (charge-coupled device) detector (a cooled CCD camera) 16, an inverted microscope 18, and a Raman optical system 20. Laser light emitted from the DPSS laser light source 12 enters an optical path via a laser optical fiber 32, and passes through a lens 202, an ND (Neutral Density) filter 204, a shutter 205, and a band-pass filter 206. Thereafter, the laser light is reflected by a flat mirror 208 and a dichroic mirror 210, and arrives at a biaxial Galvano mirror 30 which comprises Galvano mirrors arranged on an X axis and a Y axis that are orthogonal to each other. By rotating the biaxial Galvano mirror 30 at high speed, laser light can be applied in a turning manner to a region that has been predetermined in a bright field image and includes a cell 50. Further, it is possible to adopt, as laser light outputted from the DPSS laser light source 12, various kinds of laser light such as 266 nm (nanometer) (a Deep UV laser), 320 nm (a DPSS laser), 325 nm (a He—Cd laser), 442 nm (a He—Cd laser), 447 nm (an air cooled Ar+ laser), 448 nm (an air cooled Ar+ laser), 514 nm (an air cooled Ar+ laser), 532 nm (a DPSS laser), 633 nm (a He—Ne laser), 785 nm (a near-infrared semiconductor laser), 839 nm (an air cooled semiconductor laser), 1064 nm (an air cooled DPSS laser), and so on, and the kind of the laser light can selected appropriately by a person skilled in the art.

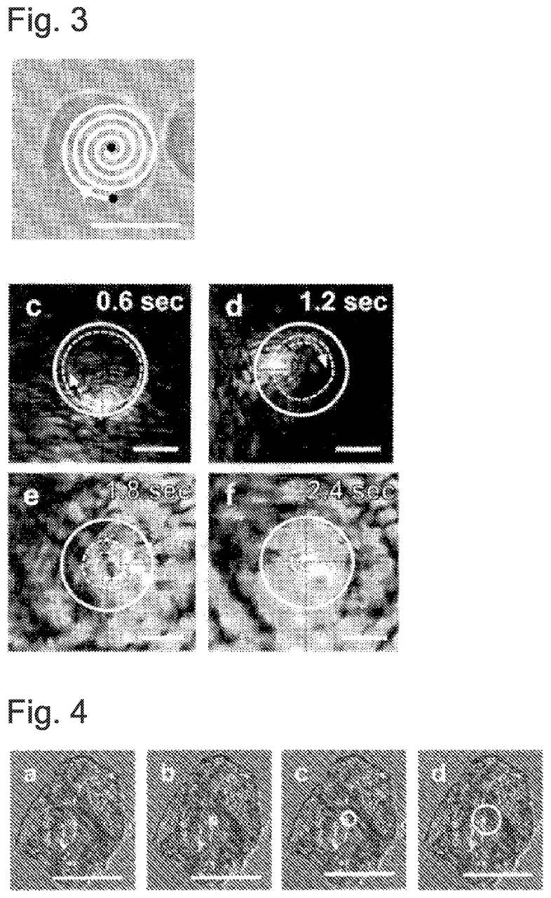

FIG. 3 is a figure showing examples of scenes wherein approximately circular specific regions in cells are irradiated by laser light. As shown in FIG. 3, with respect to each of individual cells (with respect to a single cell), it is possible to irradiate a region in the cell with laser light. When performing measurement of each of cells, any region in a cell can be designated as an irradiation region (an irradiation area). For example, a largest available region can be designated as the irradiation region. For example, in the case that a circular cell having a diameter of 8-15 μm (a human T cell or the like) is selected as an object of measurement, a circular region having a diameter of 10 μm may be selected as an irradiation region. Especially, since Raman scattered light generated from a nucleus region and that of the Raman scattered light generated from a region other than the nucleus region (cytoplasm) are different from each other, it is preferable to designate, as a measurement region, a region that covers the nucleus and the cytoplasm. By adopting the above construction, the present system integrates Raman scattered light originated in a nucleus with Raman scattered light originated in cytoplasm, and makes it possible to detect the above Raman scattered light as a single Raman signal. In a different example, it is possible to designate a limited region in a cell as an irradiation region. For example, in the case that an adhered cell is selected as an object of measurement, it is also possible to limit a region, that is to be designated as an irradiation region, to that where a nucleus exists. Further, it is possible to limit a region, that is to be designated as an irradiation region, to that (cytoplasm) other than a region including a nucleus.

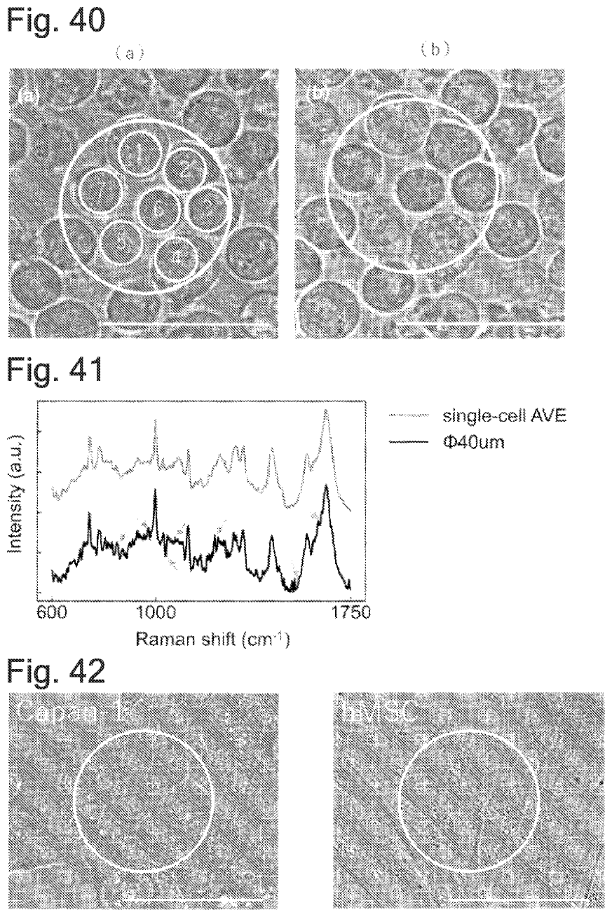

In a different construction, it is also possible to set a region, that is to be irradiated by laser light, to have a large size, and irradiate plural cells existing in the region collectively with laser light (FIG. 40 and so on). Regarding the expression “approximately circular shape,” it should be reminded that it is intended to allow the meaning of the above expression to cover a circular shape having some distortion (a case that the shape is almost circular), in addition to a completely circular shape (perfect circle). Further, although the Raman spectroscopy system 1 according to the present embodiment emits laser light to an approximately circular region including one or plural cells in FIG. 3, it is also possible to emit laser light to a quadrangle region (for example, any one of various kinds of quadrangles such as a square, a rectangular, a rhombus, a parallelogram, a trapezoid, or the like may be adopted) including one or plural cells. Further, the Raman spectroscopy system 1 can emit, in a turning manner, laser light to a region that has any one of shapes such as a triangle shape, a hexagon shape, and so on, and includes one or plural cells. Further, the Raman spectroscopy system 1 according to the present embodiment may emit laser light to any one of various shaped region including one or plural cells in such a manner that the position irradiated with the laser light moves from an outer side to an inner side of the region (i.e., from an outer periphery to a center in the region) in a turning manner, or moves from the inner side to the outer side of the region in a turning manner. Further, the Raman spectroscopy system 1 according to the present embodiment may emit laser light to any one of various shaped region including one or plural cells in such a manner that the position irradiated with the laser light moves, in a turning manner in a rotation direction, from an outer side to an inner side of the region and finally to a position close to the center of the region, and, thereafter, moves, in a turning manner to move spirally in the above rotation direction or a reverse rotation direction, from the inner side to the outer side of the region; and, further, it may be possible to repeat the above laser light emitting process, i.e., the process for moving the position irradiated with the laser light, in a turning manner, from the outer side to the inner side and thereafter from the inner side to the outer side of the region. By the above construction, the shaped region can be irradiated fully and sufficiently. Further, the rotation direction at the time when the laser light is emitted in a turning manner may be a clockwise rotation direction or a counterclockwise rotation direction. Further, the intervals in the spiral, that is a track of the turning-manner movement, can be adjusted appropriately. It should be reminded that the expression “in a turning manner (emitting laser light in a turning manner)” herein means action to emit laser light as if a spiral is drawn thereby.

Further, regarding the Raman spectroscopy system 1 according to the present embodiment, the region that can be irradiated by the laser light is, for example, an approximately circular region having a radius of 0.25-150 μm (micrometer) or a quadrangle region having a minor axis (the X-axis direction or the Y-axis direction of the biaxial Galvano mirror 212) of 0.5-300 μm, the marking speed is 0.002-2 mm/ms (millimeter/millisecond), and so on. The moving speed of the laser light can be controlled by controlling the vibration speed of the biaxial Galvano mirror 30.

Here, FIG. 1 is referred to again: The scattered light from the cell 50, which has been irradiated by the laser light, propagates through the path, which is the same as the path through which the inputted laser light has passed, in an opposite direction and passes through the dichroic mirror 210 again. Further, the scattered light passes through the high-pass filter 212, wherein only the high frequency components of the scattered light pass through the filter. As a result, only the Raman scattered light is converged, via the lens 214, the shutter 215, and the optical fiber 34, to be inputted to the spectrometer 14. The Raman scattered light is detected as a Raman spectrum by the CCD detector 16, wherein the Raman spectrum is defined by a horizontal axis representing the Raman shift (wavenumber, cm⁻¹) and a vertical axis representing the intensity of the scattered light (a.u.).

### (Construction of Raman Spectroscopy System)

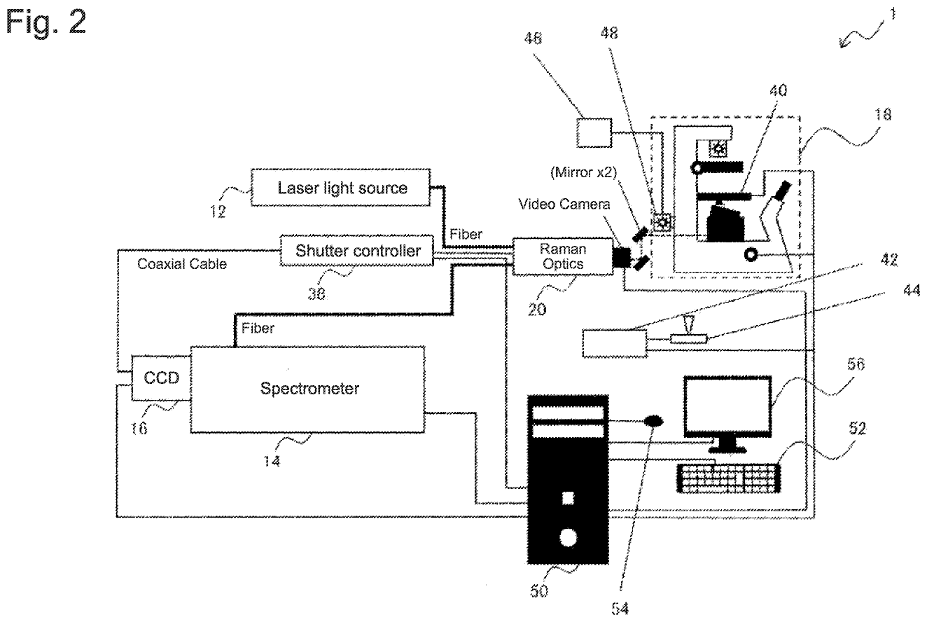

FIG. 2 is a figure which shows an example of a construction of a Raman spectroscopy system according to an embodiment of the present invention. It should be reminded that, in the present construction example, a reference symbol that is the same as that assigned to a component in FIG. 1 is assigned to a component which is the same as or equivalent to the component in FIG. 1. The Raman spectroscopy system 1 according to the present embodiment is constructed in such a manner that it comprises a DPSS laser light source 12, a spectrometer 14, a CCD detector 16, an inverted microscope 18, a Raman optics 20, and a shutter controller 38. The shutter controller 38 is a controller for controlling the shutter 205 and the shutter 215 shown in FIG. 1. Further, the Raman spectroscopy system 1 may comprise a joystick 44 which is manipulated by a user for adjusting the stage 40 of the inverted microscope 18, and a stage controller 42 for controlling the stage of the inverted microscope 18 in response to movement of the joystick 44. Further, the Raman spectroscopy system 1 may comprise an epi-illumination controller 46 for controlling epi-illumination device 48 for the inverted microscope 18.

Also, the Raman spectroscopy system 1 may further comprise a computer device 50, and input/output interfaces such as a keyboard 52, a mouse 54, and so on and a monitor device 15 for manipulation of the computer device 50 by a user. The computer device 50 may obtain spectrum data of a cell detected by the CCD detector 16, and display the spectrum data in the form of a graph or the like by the monitor device 56. Further, the computer device 50 can execute machine learning software. The machine learning software may be unsupervised or supervised machine learning software that receives spectrum data as input and outputs answers such as those relating to classification of cells and so on, as will be explained later. The computer device 50 can be realized by using a hardware construction similar to that of a general computer device.

The computer device 50 may comprise, for example, a processor, a RAM (Random Access Memory), a ROM (Read Only Memory), a built-in hard disk device, a removable memory such as an external hard disk device, a CD, a DVD, a USB memory, a memory stick, a SD card, or the like, an input/output user interface (a touch panel, a speaker, a microphone, a lamp, or the like), a wired/wireless communication interface which can communicate with another computer device, and so on. For example, the processor in the computer device 50 may read various kinds of programs such as a spectrum data analyzing program, a machine learning program, and so on, that have been stored in a hard disk device, a ROM, a removable memory, or the like, and put them into a memory such as a RAM or the like; and execute the programs while reading, from a hard disk device, a ROM, a removable memory, or the like, spectrum data of a cell that were detected by the CCD detector 16.

According to the Raman spectroscopy system and the spectrum data obtaining method explained above, a Raman spectrum in a wide region can be obtained in a short period of time. By adopting the above construction, laser light can be emitted efficiently and evenly while suppressing damage to a cell.

### (Method for Classifying Cells)

The present invention also provides a method for classifying cells. The cell classification method comprises, as explained above, a step of classifying a cell based on spectrum data obtained from the cell. In the present case, cell classification includes classification of the kinds of cells and classification of the states of cells. The states of cells include a physiological state of a cell, an activated state of a cell (due to an endogenous or exogenous stimulator, a signal transduction substance, a compound other than the above, or the like), and so on. As the above states, states of cell division, cell death, cell neoplastic transformation, cell differentiation, cell dedifferentiation, metabolic state change, form or shape change, cell infection due to a foreign cell (an infectious cell such as a virus, a bacteria, or the like), and so on can be listed, and it should be reminded that the states are not limited to those listed above. Further, a cell may be in any of a floating state and an adhered state.

Cells which are to be used as objects of analysis are animal cells, plant cells, microbial cells, and so on, i.e., the cells are not limited specifically. As animal cells, cells originated in creatures in mammals, birds, reptiles, amphibians, fishes, and so on can be listed, and, preferably, the cells are those originated in a human, a dog, a mouse, a rat, a cat, a horse, a goat, a sheep, a bovine, a flog, and so on. For example, the cells to be listed are those included in undifferentiated cells, multipotent cells, pluripotent cells, differentiated cells, germ cells, and somatic cells, and it should be reminded that the cells are not limited to those listed above. More specifically, the cells to be listed are Jurkat cells (the human T cell line), mouse embryonic fibroblasts (MEFs), embryonic stem cells, immortalized human adipose-derived mesenchymal stem cells (hMSCs), human induced pluripotent stem cells (hiPSCs), and so on, and it should be reminded that the cells are not limited to those listed above. As plant cells, cells originated in angiosperms, gymnosperms, pteridophytes, bryophytes, algae, and so on can be listed, and, preferably, the cells are those originated in vegetables, flowers, trees, and so on. The microbial cells can be any of those in prokaryotes and eukaryotes. For example, the cells which can be listed are those in colon bacilli, Staphylococcus, halophilic bacteria, thermophilic bacteria, yeast, fungi, bacteria, and so on, and it should be reminded that the cells are not limited to those listed above.

The step of classifying a cell comprises a step of comparing spectrum data obtained from a cell, which is an object of analysis, with standard spectrum data. In this regard, the standard spectrum data is Raman spectrum data obtained from a cell with respect to which the type or the state has been known. The standard spectrum data may be obtained at any timing. For example, it may be obtained during a stage before analysis, during analysis, or after analysis.

The standard spectrum data are obtained with respect to each of different kinds of cells or each of cells in different states. Further, in the case that standard spectrum data of each of cells include a characteristic spectrum region, it is preferable that the characteristic spectrum region be specified. There may be one or more characteristic spectrum regions. Further, in the case that plural characteristic spectrum regions exist, it is possible to perform analysis of a cell by using all of the characteristics; however, it is also possible to perform analysis of the cell by selecting a highly relevant characteristic and using it. A person skilled in the art can perform selection of such a spectrum in an appropriate manner.

Further, the step of classifying a cell can be performed by using machine learning. Machine learning may be any of supervised machine learning and unsupervised machine learning. By using machine learning, learning from data, that are obtained as a result of analysis, is performed autonomously; so that it is expected that accuracy of classification of a cell increases as the number of repetitions of analysis increases.

The present invention will be explained more specifically with reference to the embodiments explained below. It should be reminded that the embodiments are those provided to understand the present invention, and are not those intended to be used for limiting the scope of the present invention.

### EMBODIMENTS

### (Tangible Construction of Raman Spectroscopy System)

The inventors have performed measurement of cells by using a Raman spectroscopy system according to a present embodiment. In above measurement, the Ti2-U microscope available from Nikon Corporation was used as the inverted microscope 18. As a means for excitation, a DPSS 532-nm green laser (DL 532-100: maximum output, 120 mW (milliwatt)) coupled to an optical fiber was used. As the objective lens, Lambda 40×C/NA 0.95 (Nikon Corporation, CFI Plan Apo Lambda) was used in a sample stage in a state that laser light having power of approximately 40 mW was being outputted, wherein the laser spot size was set to that less than 450 nm. The backscattered Raman signal was dispersed by a spectrometer comprising a 1200 g grating (AIRIX Corp., STR200-2LC), and detected by using a CCD camera (Andor Technology Limited, iVac316). The detectable Raman spectrum region was 46-3110 cm⁻¹. Further, for allowing laser light to move in a specific region, a biaxial Galvano mirror was arranged in a laser light path.

### (Collecting of Raman Data)

For calibration of the spectrometer, a spectrum of sulfur, that is a Raman shift standard, was measure before sample measurement. The spectrum of sulfur was detected by using a point irradiation method, by using an ND (Neutral Density) filter 1 or 5% and by setting exposure time to 1 second. For correction, detected three characteristic spectrum peaks were adjusted to correspond to 153.8, 219.1, and 473.2 cm⁻¹ (ASTME1840-96, DOI: 10.1520/E1840-96).

(Comparison Between Raman Spectrum Obtained by Performing Point Method and that Obtained by Performing Irradiation Method According to Present Embodiment)

A prior-art point irradiation (point) method and an irradiation method performed by the Raman spectroscopy system 1 according to the present embodiments were compared with each other.

(1) Comparison in the Case when Palmitic Acid is Used as Analysis Subject

High-purity (<95%) solid palmitic acid was used as an analysis subject. The spectrum of the solid palmitic acid was obtained by performing point irradiation (the laser spot diameter <450 nm) (a in FIG. 4). On the other hand, regarding the case of the irradiation method according to the present embodiment, the spectra of the solid palmitic acid were obtained in circular regions having diameters of 5, 10, and 20 μm (b-d in FIG. 4). The exposure time of the laser in the case of each of the irradiation methods was set to 1 second, and the oscillation speed of each mirror of the biaxial Galvano mirror 30 was adjusted to make the laser possible to illuminate the whole region of a circle even if the circle has a diameter of 20 μm (1 mm/ms).

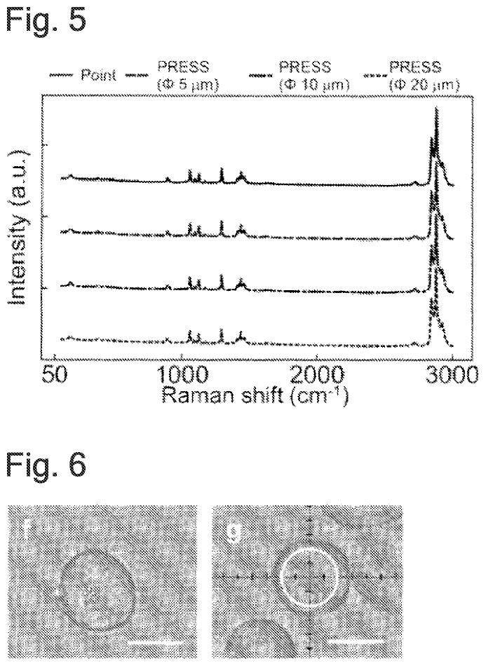

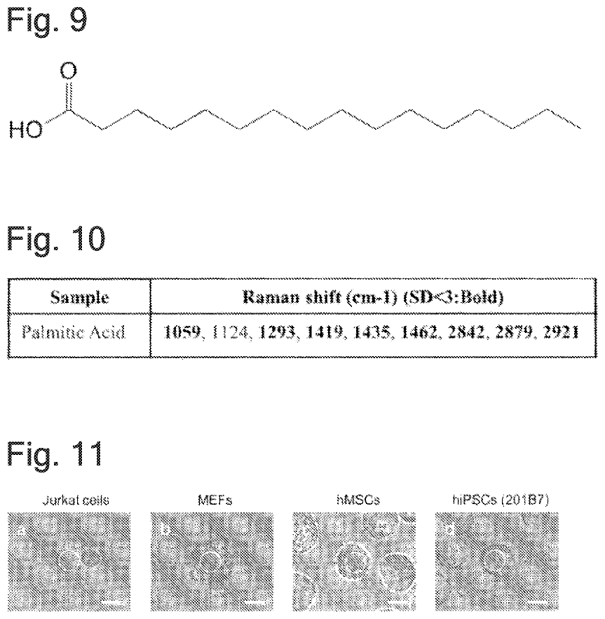

With respect to all the methods, peaks based on the palmitic acid were found at 1059, 1124, 1293, 1419, 1435, 1462, 2842, 2879, and 2921 cm⁻¹ (FIG. 5). As a result that the above spectrum was collated with the spectral database provided by Wiley Science solutions (KnowItAll), it was confirmed that it coincides with the spectrum of the palmitic acid (code: FFRX #478) (FIGS. 8-10). Accordingly, it was proved that the irradiation method according to the present embodiment generated, without degrading the intensity, spectrum data comparable to those obtained by performing the conventional point irradiation method.

(2) Comparison in the Case when Living Cells are Used as Analysis Subjects

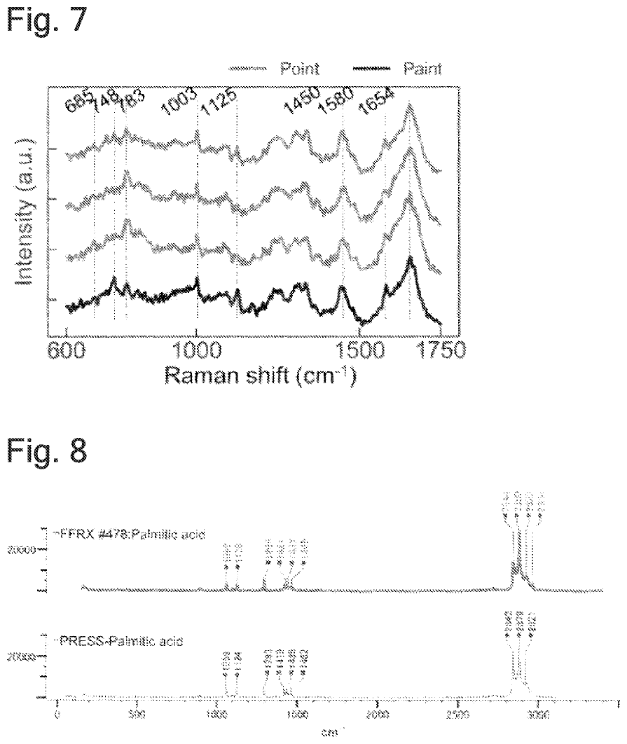

Next, Raman measurement of living cells, that was performed according to the irradiation method according to the present embodiment, was tested by using Jurkat cells of the human T cell line. In the present irradiation method, the exposure time was set to 3 seconds, and the spectrum was measured in a circular region having a diameter of 10 μm in a cell (b in FIG. 6). On the other hand, in the case that the prior-art point irradiation method was used and the exposure time was set to that equal to that in the above case, i.e., 3 seconds, three spectra were obtained in random positions (a in FIG. 6). It was shown, with respect to both the irradiation method according to the present embodiment and the point irradiation method, that there are characteristic peaks of nucleic acid (685, 748, 783 cm⁻¹), protein (1003, 1450 cm⁻¹), lipid (1125, 1580, 1654 cm⁻¹), and so on in the fingerprint region of 600-1800 cm⁻¹ (FIG. 7). The spectra obtained by performing the irradiation method according to the present embodiment did not show any decrease in sensitivity compared with those obtained by performing point irradiation. Three spectra obtained by point irradiation had some Raman shift regions having different intensities, such as 748 cm⁻¹, 1125 cm⁻¹, and so on.

### (Classification of Cell Types Using Irradiation Method According to Present Embodiment)

For confirming whether cell types can be classified by using the irradiation method according to the present embodiment, spectra were obtained from Jurkat cells, mouse embryonic fibroblasts (MEFs), immortalized human adipose-derived mesenchymal stem cells (hMSCs), and human induced pluripotent stem cells (hiPSCs).

### (1) Cells in Floating States

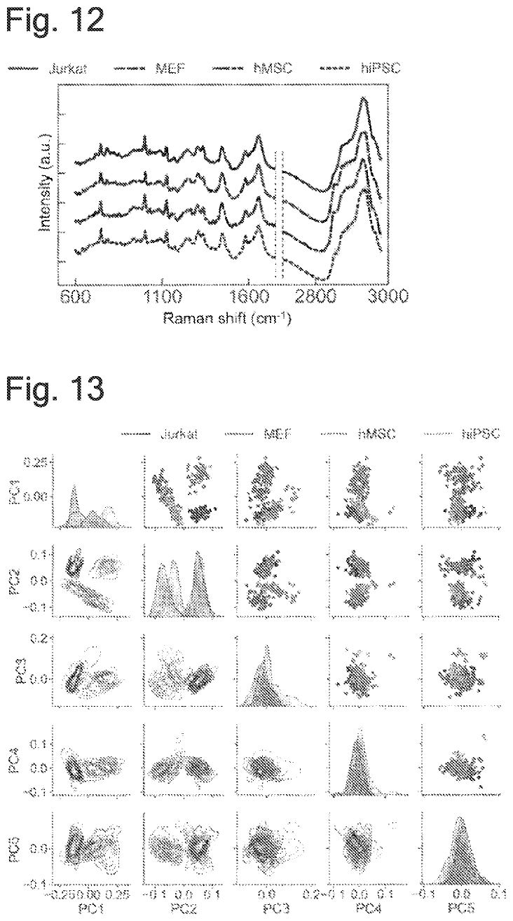

MEFs, hMSCs and hiPSCs, i.e., except Jurkat cells, are usually cultured in adhered states on culture dishes. For matching each of the states of cells to that of the Jurkat cell, the cells were processed by using Tryp^{LE} Express Enzyme (registered trademark) or Accutase (the both are available from Thermo Fisher Scientific, Waltham, MA, USA), and measured when they were in floating states. With respect to each cell, Raman spectrum was obtained by performing measurement in a circular region having a diameter of 10 μm. Measurement performed during the floating state is shown in FIG. 11. In the floating states, 50 cells for each cell type were measured (a total of 200 samples). The obtained spectra were smoothed, baseline corrected, and normalized. Average spectrum data of respective cells are shown in FIG. 12.

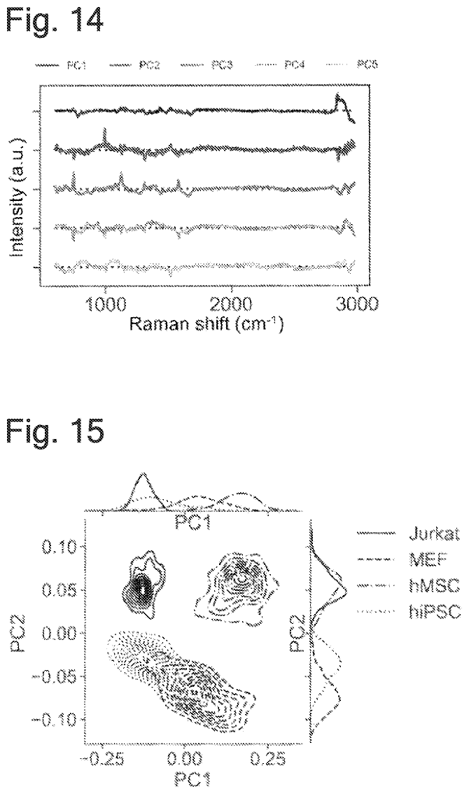

For examining discrimination of respective cell types, spectra that have been obtained as multivariate data for principal component analysis (PCA), that is an unsupervised machine learning method, were used. First five principal components (PCs) that contribute to dispersion of all cells were extracted by the PCA, and distribution of cells with respect to the five PCs were plotted in the form of a pair plot or showed in the form of kernel density estimation distribution (FIG. 13). Contribution of the five PCs to the cell dispersion in the floating states were 64.89%, 10.52%, 3.86%, 2.83%, and 1.63%. The quantities of loads with respect to the wavenumbers of the five PCs, with respect to cell dispersion during the floating states, are shown in the form of graphs (FIG. 14). With respect to PC1 and PC2 in the floating states, visual classification of them could be made (FIG. 15). The Jurkat cells, MEFs, hMSCs, and iPSCs showed different cell distribution densities. Especially, in the floating states, it was shown that PC1 contributes to classifying of the four species.

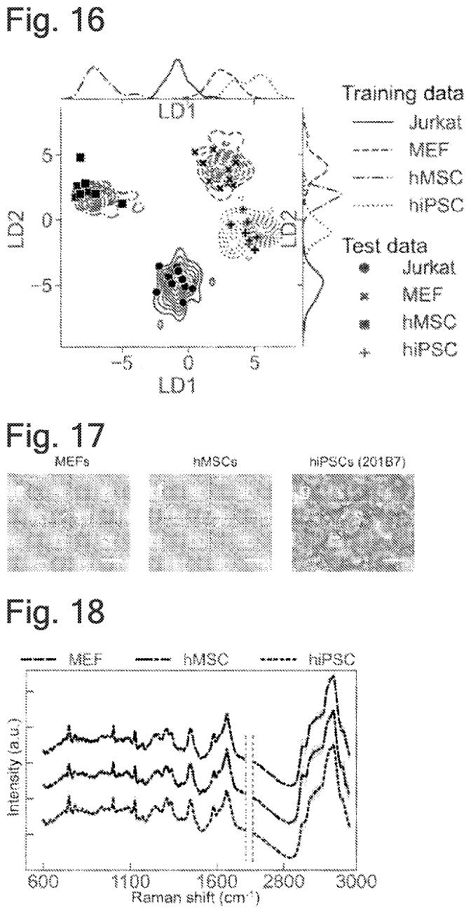

Next, classification was validated by supervised machine learning method. With respect to the above classification, a linear discriminant analysis (LDA) that is a supervised learning technique, was used. The LDA is a method to create classification axes by extracting features between classes and optimize separation of the classes. For testing the accuracy of classification, all spectrum data (200 floating-state cells) were randomly divided to training data accounting for 80% and test data accounting for 20%. The LDA defined, by using the training data, new three or two classification axes (the number of extracted axes is that obtained by subtracting 1 from the number of classes) that could best classify the cell types. Next, by applying the test data to the extracted axes, accuracy of classification was tested. FIG. 16 shows, in terms of respective cell types, distribution of cells with respect to LD1 and LD2 axes. The cells in the training data used to extract the classification axes are shown in the form of kernel density estimation distribution, and the cells used in the test data are shown in the form of a plot. The LDA classified the four cell types more clearly than the PCA, and the cells in the test data 100%-matched the original labels. By adopting machine learning to combine it, it has become possible to classify the cell types in the floating conditions more accurately.

### (2) Cells in Adhered States

With respect to the MEFs, hMSCs and hiPSCs, measurement of them was also performed in the states that they were adhered to culture dishes (FIGS. 17-22). In the adhered states, 100 cells for each cell type were measured (a total of 300 samples). The obtained spectra were smoothed, baseline corrected, and normalized. Average spectrum data of respective cells are shown in FIG. 18. It was possible to classify cell types in the states of adhesive culturing.

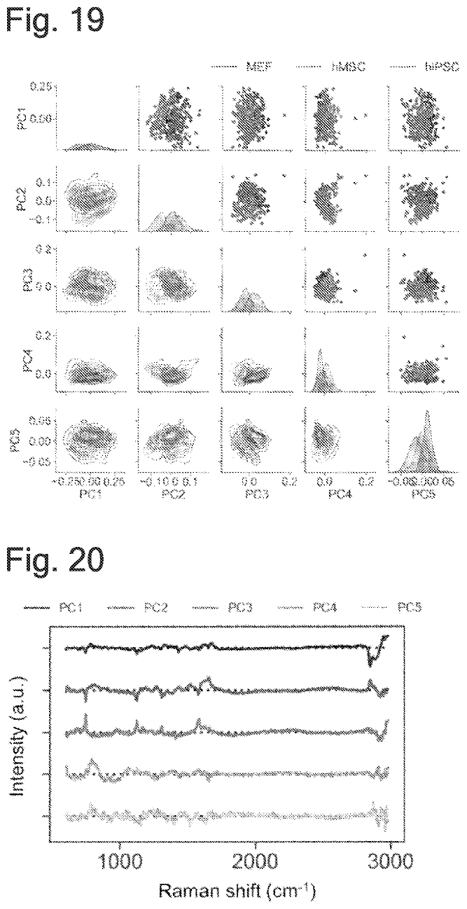

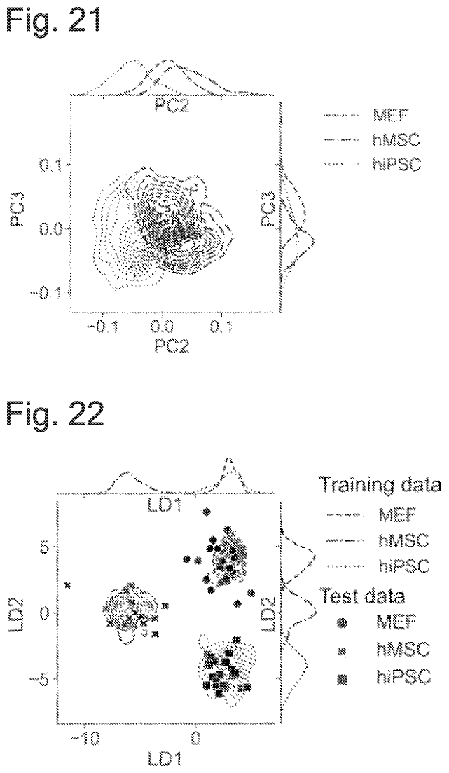

For examining discrimination of respective cell types, spectra that have been obtained as multivariate data for principal component analysis (PCA), that is an unsupervised machine learning method, were used. First five principal components (PCs) that contribute to dispersion of all cells were extracted by the PCA, and distribution of cells with respect to the five PCs were plotted in the form of a pair plot or showed in the form of kernel density estimation distribution (FIG. 19). Contribution of the five PCs to the cell dispersion in the adhered states were 49.7%, 12.3%, 7.16%, 3.93%, and 1.69%. The quantities of loads with respect to the wavenumbers of the five PCs, with respect to cell dispersion during the adhered states, are shown in the form of graphs (FIG. 20). With respect to PC2 and PC3 in the adhered states, visual classification of them could be made most preferably (FIG. 21). The MEFs, hMSCs, and iPSCs showed different cell distribution densities. Especially, PC2 contributed to classification of the hMSCs and iPSCs, and PC2 contributed to classification of MEFs and hMSCs.

Next, classification was tested by performing training with respect to the spectrum data by using machine learning. With respect to the above classification, a linear discriminant analysis (LDA) that is a supervised learning technique, was used. The LDA is a method to create classification axes by extracting features between classes and optimize separation of the classes. For testing the accuracy of classification, all spectrum data (300 adhered-state cells) were randomly divided to training data accounting for 80% and test data accounting for 20%. The LDA defined, by using the training data, new three or two classification axes (the number of extracted axes is that obtained by subtracting 1 from the number of classes) that could best classify the cell types. Next, by applying the test data to the extracted axes to test accuracy of classification. FIG. 22 shows, in terms of respective cell types, distribution of cells with respect to LD1 and LD2 axes. The cells in the training data used to extract the classification axes are shown in the form of kernel density estimation distribution, and the cells used in the test data are shown in the form of a plot. The LDA classified the three cell types more clearly than the PCA, and the cells in the test data 100%-matched the original labels. By adopting machine learning to combine it, it has become possible to classify the cell types in the adhesive cultivation states more accurately. (Classification of Same Cells in Different States)

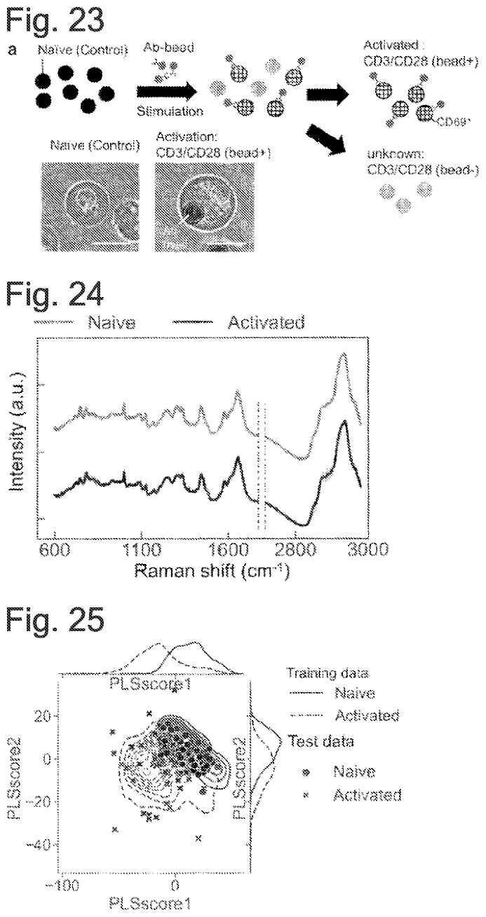

It was examined whether same cell types in different states can be discriminated by using spectra obtained by performing the irradiation method according to the present embodiment. Jurkat cells (the human T cell line) were stimulated to obtain activated cells. The above cells and naive cells were analyzed. For stimulating the cells, magnetic beads bonded with CD3 and CD28 antibodies were used (FIG. 23). For confirming activation of the cells, qPCR was performed. It was confirmed with respect to the activated cells that gene expression level of the activation markers IL-2 and TNF-α had increased, compared with that relating to the naive cells.

300 spectra were obtained from the activated cells and the naive cells, respectively, by performing the present irradiation method. For avoiding effect of the magnetic bead on the spectrum, the spectrum was measured in a circular region having a diameter of 5 μm. With respect to each of the activated cell and the naive cell, averages of spectrum data in the region of the Raman shift of 600-2980 cm⁻¹ are shown in FIG. 24.

Next, the spectrum data were applied to the supervised machine learning and an attempt to clearly classify the activation states was made. For achieving highly accurate classification of the activated cells and the naive cells, the linear discriminant analysis (LDA), partial least squares-discriminant analysis (PLS-DA) based on discriminant analysis, multilayer perceptron (MLP) based on artificial neural networks (ANN), and support vector machine (SVM) were tested.

For supervised machine learning, all data of the activated cells and the naive cells were randomly divided to training data accounting for 80% and test data accounting for 20%. Axes for classifying the two cell types by using four classification methods were extracted by using the training data, and the test data were classified in relation to the above defined axes. A false positive ratio (FPR) and a true positive rate (TPR) were calculated from the original label and the classified label of the test data, and receiver operating characteristic (ROC) curves were created by using the FPR and the TPR. Further, an area under the ROC curve (AUC) that represents classification accuracy, sensitivity, and specificity, and a root mean square error (RMSE) (Table 1) were calculated. Table 1 shows average values that were obtained after repeating the classification test ten times.

TABLE 1

| Method | Sensitivity | Specificity | AUC | RMSE |
|---|---|---|---|---|
| LDA | 0.96 | 0.91 | 0.93 | 0.25 |
| PLS-DA | 0.92 | 0.99 | 0.95 | 0.21 |
| SVM | 0.97 | 0.98 | 0.97 | 0.15 |
| MLP | 0.98 | 0.98 | 0.98 | 0.14 |

AUCs of the LDA, PLS-DA, SVM and MLP were 0.93, 0.95, 0.97, and 0.98, respectively, and all of them were judged to be high. By using PLS-DA that is used as a dimensionality reduction method, two kinds of feature quantities that contribute to classification of the states of the cells were extracted as PLS scores. By using scores 1 and 2 that were used for extracting activated cells and naive cells, the training data were shown in the form of a kernel density estimation distribution. Thereafter, the test data (represented by dots) were superimposed on the training data (FIG. 25). By adopting the supervised machine learning method to combine it, it has become possible to classify activated cells and naive cells more accurately.

(Classification of Same Cells in Different States (Stimulation with Refined CD3/CD28 Antibodies))

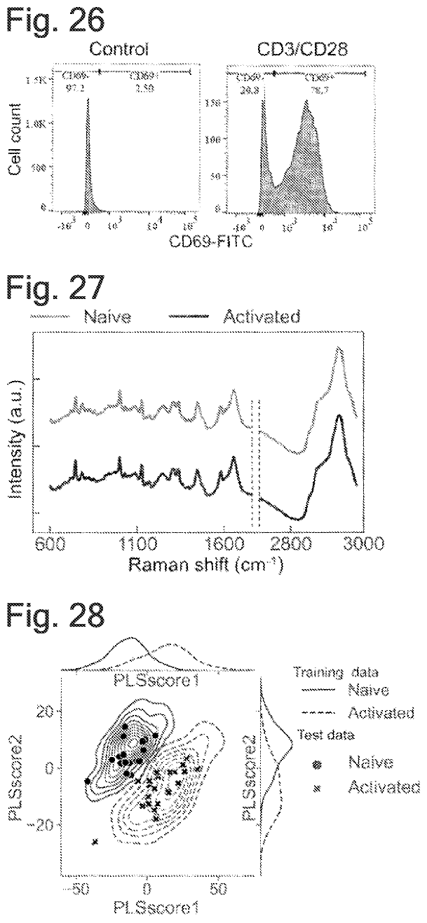

It was examined whether same cell types in different states can be discriminated by using spectra obtained by performing the irradiation method according to the present embodiment. Jurkat cells (the human T cell line) were stimulated to obtain activated cells. The above cells and naive cells were analyzed. For stimulating the cells, refined CD3 and CD28 antibodies were used. In this regard, the “refined CD3 and CD28 antibodies” mean CD3 antibodies and CD28 antibodies which are free (i.e., to which magnetic beads are not bonded). For confirming activation of the cells, a positive ratio of activation markers CD69 was measured by using a flow cytometer (FIG. 26). It was confirmed with respect to the cells after stimulation that the positive ratio of CD69 had increased, compared with that relating to the controls (the naive cells).

100 spectra were obtained from the activated cells and the naive cells, respectively, by performing the present irradiation method. The spectrum was measured in a circular region having a diameter of 5 μm. With respect to each of the activated cell and the naive cell, averages of spectrum data in the region of the Raman shift of 600-2980 cm⁻¹ are shown in FIG. 27.

Next, the spectrum data were applied to the supervised machine learning and an attempt to clearly classify the activation states was made. For achieving highly accurate classification of the activated cells and the naive cells, the linear discriminant analysis (LDA), partial least squares-discriminant analysis (PLS-DA) based on discriminant analysis, multilayer perceptron (MLP) based on artificial neural networks (ANN), and support vector machine (SVM) were tested.

For supervised machine learning, all data of the activated cells and the naive cells were randomly divided to training data accounting for 80% and test data accounting for 20%. Axes for classifying the two cell types by using four classification methods were extracted by using the training data, and the test data were classified in relation to the above defined axes. A false positive ratio (FPR) and a true positive rate (TPR) were calculated from the original label and the classified label of the test data, and receiver operating characteristic (ROC) curves were created by using the FPR and the TPR. Further, an area under the ROC curve (AUC) that represents classification accuracy, sensitivity, and specificity, and a root mean square error (RMSE) (Table 2) were calculated. Table 2 shows average values that were obtained after repeating the classification test ten times.

TABLE 2

| Method | Sensitivity | Specificity | AUC | RMSE |
|---|---|---|---|---|
| LDA | 0.99 | 1.00 | 0.99 | 0.05 |
| PLS-DA | 1.00 | 1.00 | 1.00 | 0 |
| SVM | 1.00 | 1.00 | 1.00 | 0 |
| MLP | 0.99 | 0.99 | 0.99 | 0.03 |

AUCs of the LDA, PLS-DA, SVM and MLP were 0.99, 1.00, 1.00, and 0.99, respectively, and all of them were judged to be high. By using PLS-DA that is used as a dimensionality reduction method, two kinds of feature quantities that contribute to classification of the states of the cells were extracted as PLS scores. By using scores 1 and 2 that were used for extracting activated cells and naive cells, the training data were shown in the form of a kernel density estimation distribution. Thereafter, the test data (represented by dots) were superimposed on the training data (FIG. 28). By adopting the supervised machine learning method to combine it, it has become possible to classify activated cells and naive cells more accurately. (Classification of Same Cells in Different States (Stimulation with PMA and A23187))

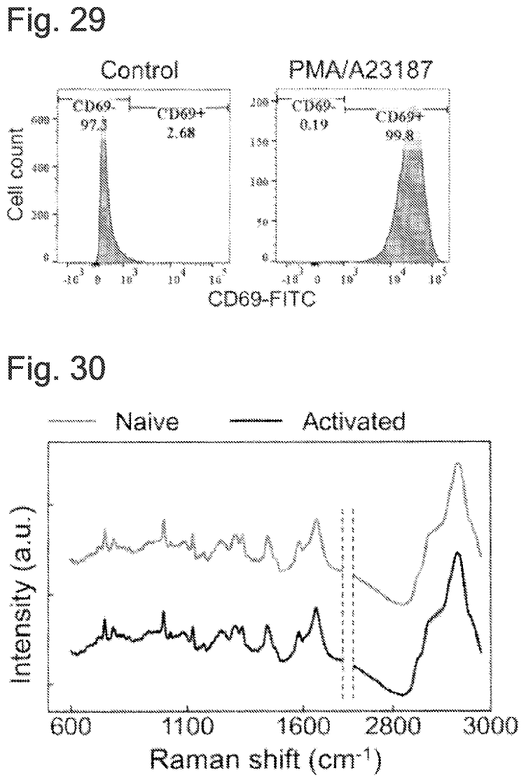

It was examined whether same cell types in different states can be discriminated by using spectra obtained by performing the irradiation method according to the present embodiment. Jurkat cells (the human T cell line) were stimulated to obtain activated cells. The above cells and naive cells were analyzed. Also, the cells were stimulated with PMA (phorbol 12-myristate 13-acetate (which has been known as a tumor promoter), 20 ng/ml) and calcium ionophore A23187 (1 μM). Thereafter, with respect to the processed cells, a positive ratio of activation markers CD69 was measured by using a flow cytometer (FIG. 29). It was confirmed with respect to the cells after stimulation that the positive ratio of CD69 was approximately 100%.

100 spectra were obtained from the activated cells and the naive cells, respectively, by performing the present irradiation method. The spectrum was measured in a circular region having a diameter of 10 μm. With respect to each of the activated cell and the naive cell, averages of spectrum data in the region of the Raman shift of 600-2980 cm⁻¹ are shown in FIG. 30.

Next, the spectrum data were applied to the supervised machine learning and an attempt to clearly classify the activation states was made. For achieving highly accurate classification of the activated cells and the naive cells, the linear discriminant analysis (LDA), partial least squares-discriminant analysis (PLS-DA) based on discriminant analysis, multilayer perceptron (MLP) based on artificial neural networks (ANN), and support vector machine (SVM) were tested.

For supervised machine learning, all data of the activated cells and the naive cells were randomly divided to training data accounting for 80% and test data accounting for 20%. Axes for classifying the two cell types by using four classification methods were extracted by using the training data, and the test data were classified in relation to the above defined axes. A false positive ratio (FPR) and a true positive rate (TPR) were calculated from the original label and the classified label of the test data, and receiver operating characteristic (ROC) curves were created by using the FPR and the TPR. Further, an area under the ROC curve (AUC) that represents classification accuracy, sensitivity, and specificity, and a root mean square error (RMSE) (Table 3) were calculated. Table 3 shows average values that were obtained after repeating the classification test ten times.

TABLE 3

| Method | Sensitivity | Specificity | AUC | RMSE |
|---|---|---|---|---|
| LDA | 0.89 | 0.97 | 0.94 | 0.25 |
| PLS-DA | 0.95 | 0.97 | 0.97 | 0.17 |
| SVM | 0.96 | 0.98 | 0.97 | 0.15 |
| MLP | 0.92 | 0.97 | 0.95 | 0.20 |

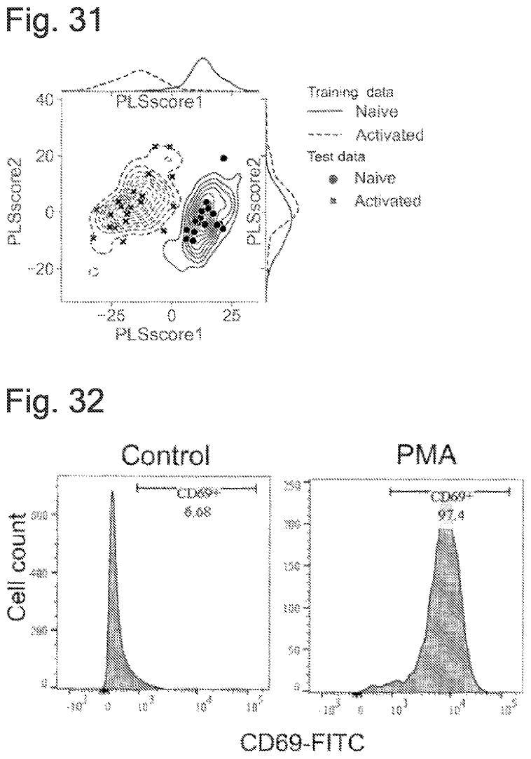

AUCs of the LDA, PLS-DA, SVM and MLP were 0.94, 0.97, 0.97, and 0.95, respectively, and all of them were judged to be high. By using PLS-DA that is used as a dimensionality reduction method, two kinds of feature quantities that contribute to classification of the states of the cells were extracted as PLS scores. By using scores 1 and 2 that were used for extracting activated cells and naive cells, the training data were shown in the form of a kernel density estimation distribution. Thereafter, the test data (represented by dots) were superimposed on the training data (FIG. 31). By adopting the supervised machine learning method to combine it, it has become possible to classify activated cells and naive cells more accurately.

(Classification of Same Cells in Different States (Stimulation with PMA))

It was examined whether same cell types in different states can be discriminated by using spectra obtained by performing the irradiation method according to the present embodiment. Jurkat cells (the human T cell line) were stimulated to obtain activated cells. The above cells and naive cells were analyzed. Also, the cells were stimulated with PMA (20 ng/ml). Thereafter, with respect to the processed cells, a positive ratio of activation markers CD69 was measured by using a flow cytometer (FIG. 32). It was confirmed with respect to the cells after stimulation that the positive ratio of CD69 was approximately 100%.

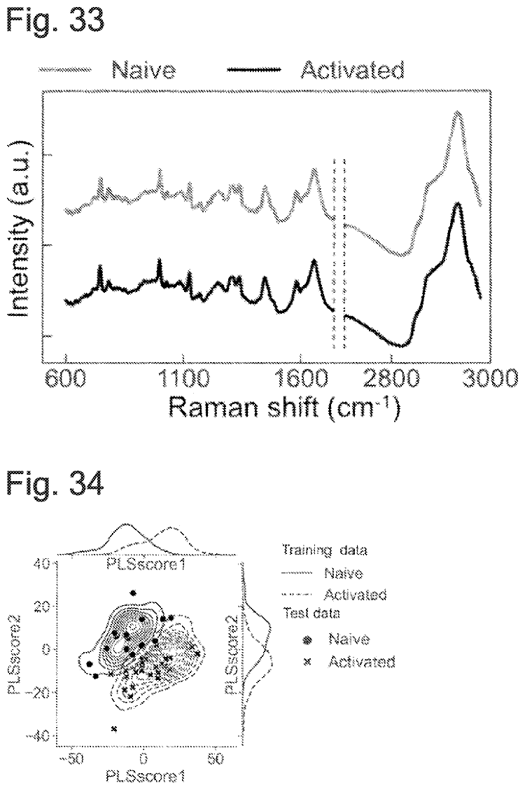

100 spectra were obtained from the activated cells and the naive cells, respectively, by performing the present irradiation method. The spectrum was measured in a circular region having a diameter of 10 μm. With respect to each of the activated cell and the naive cell, averages of spectrum data in the region of the Raman shift of 600-2980 cm⁻¹ are shown in FIG. 33.

Next, the spectrum data were applied to the supervised machine learning and an attempt to clearly classify the activation states was made. For achieving highly accurate classification of the activated cells and the naive cells, the linear discriminant analysis (LDA), partial least squares-discriminant analysis (PLS-DA) based on discriminant analysis, multilayer perceptron (MLP) based on artificial neural networks (ANN), and support vector machine (SVM) were tested.

or supervised machine learning, all data of the activated cells and the naive cells were randomly divided to training data accounting for 80% and test data accounting for 20%. Axes for classifying the two cell types by using four classification methods were extracted by using the training data, and the test data were classified in relation to the above defined axes. A false positive ratio (FPR) and a true positive rate (TPR) were calculated from the original label and the classified label of the test data, and receiver operating characteristic (ROC) curves were created by using the FPR and the TPR. Further, an area under the ROC curve (AUC) that represents classification accuracy, sensitivity, and specificity, and a root mean square error (RMSE) (Table 4) were calculated. Table 4 shows average values that were obtained after repeating the classification test ten times.

TABLE 4

| Method | Sensitivity | Specificity | AUC | RMSE |
|---|---|---|---|---|
| LDA | 0.96 | 0.97 | 0.97 | 0.16 |
| PLS-DA | 1.00 | 1.00 | 1.00 | 0 |
| SVM | 1.00 | 1.00 | 1.00 | 0 |
| MLP | 0.99 | 0.99 | 0.99 | 0.05 |

AUCs of the LDA, PLS-DA, SVM and MLP were 0.97, 1.00, 1.00, and 0.99, respectively, and all of them were judged to be high. By using PLS-DA that is used as a dimensionality reduction method, two kinds of feature quantities that contribute to classification of the states of the cells were extracted as PLS scores. By using scores 1 and 2 that were used for extracting activated cells and naive cells, the training data were shown in the form of a kernel density estimation distribution. Thereafter, the test data (represented by dots) were superimposed on the training data (FIG. 34). By adopting the supervised machine learning method to combine it, it has become possible to classify activated cells and naive cells more accurately.

(Classification of Same Cells in Different States (Stimulation with PHA))

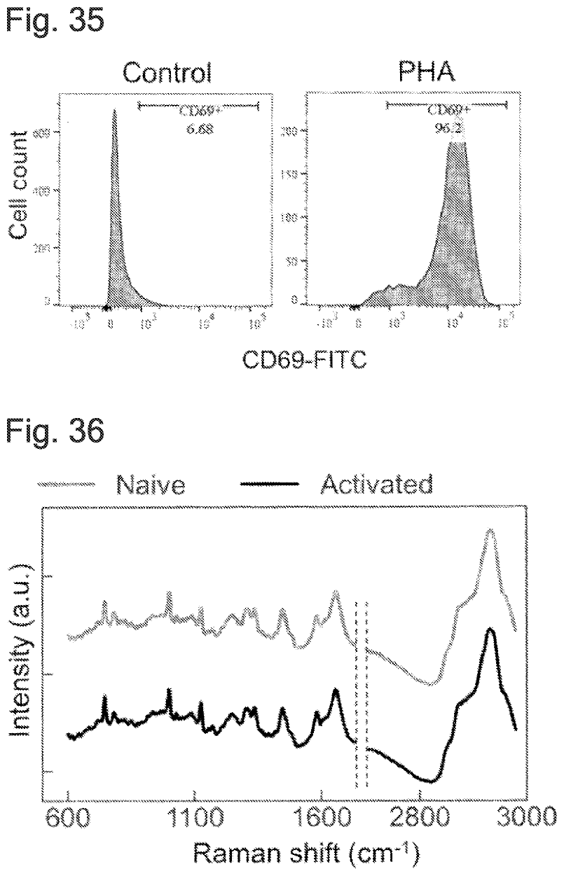

It was examined whether same cell types in different states can be discriminated by using spectra obtained by performing the irradiation method according to the present embodiment. Jurkat cells (the human T cell line) were stimulated to obtain activated cells. The above cells and naive cells were analyzed. Also, the cells were stimulated with PHA (Phytohemagglutinin, 1 μg/ml). Thereafter, with respect to the processed cells, a positive ratio of activation markers CD69 was measured by using a flow cytometer (FIG. 35). It was confirmed with respect to the cells after stimulation that the positive ratio of CD69 was approximately 100%.

100 spectra were obtained from the activated cells and the naive cells, respectively, by performing the irradiation method according to the present embodiment. The spectrum was measured in a circular region having a diameter of 10 μm. With respect to each of the activated cell and the naive cell, averages of spectrum data in the region of the Raman shift of 600-2980 cm⁻¹ are shown in FIG. 36.

Next, the spectrum data were applied to the supervised machine learning and an attempt to clearly classify the activation states was made. For achieving highly accurate classification of the activated cells and the naive cells, the linear discriminant analysis (LDA), partial least squares-discriminant analysis (PLS-DA) based on discriminant analysis, multilayer perceptron (MLP) based on artificial neural networks (ANN), and support vector machine (SVM) were tested.

For supervised machine learning, all data of the activated cells and the naive cells were randomly divided to training data accounting for 80% and test data accounting for 20%. Axes for classifying the two cell types by using four classification methods were extracted by using the training data, and the test data were classified in relation to the above defined axes. A false positive ratio (FPR) and a true positive rate (TPR) were calculated from the original label and the classified label of the test data, and receiver operating characteristic (ROC) curves were created by using the FPR and the TPR. Further, an area under the ROC curve (AUC) that represents classification accuracy, sensitivity, and specificity, and a root mean square error (RMSE) (Table 5) were calculated. Table 5 shows average values that were obtained after repeating the classification test ten times.

TABLE 5

| Method | Sensitivity | Specificity | AUC | RMSE |
|---|---|---|---|---|
| LDA | 0.89 | 0.97 | 0.94 | 0.25 |
| PLS-DA | 0.95 | 0.99 | 0.97 | 0.17 |
| SVM | 0.96 | 0.98 | 0.97 | 0.15 |
| MLP | 0.92 | 0.97 | 0.95 | 0.20 |

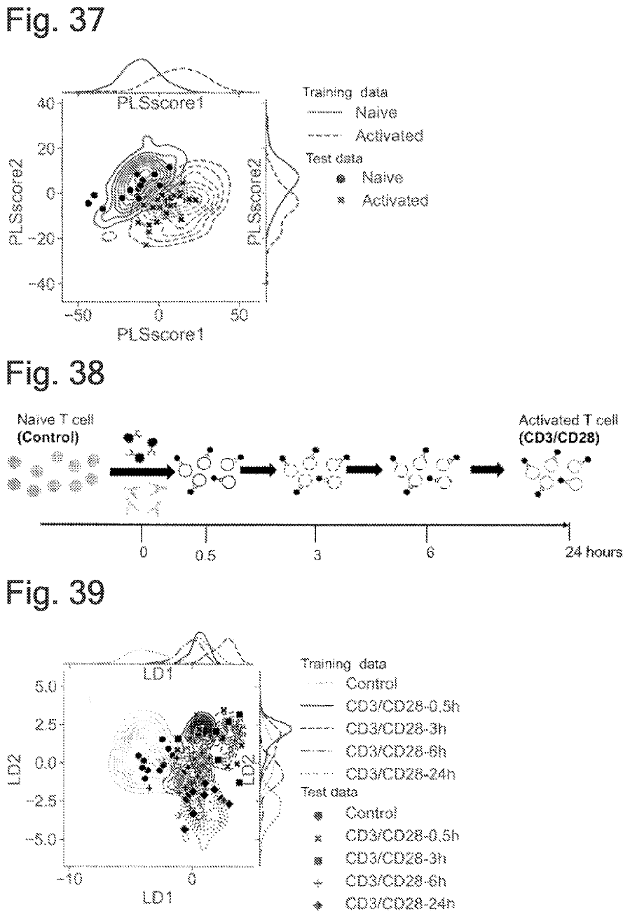

AUCs of the LDA, PLS-DA, SVM and MLP were 0.94, 0.97, 0.97, and 0.95, respectively, and all of them were judged to be high. By using PLS-DA that is used as a dimensionality reduction method, two kinds of feature quantities that contribute to classification of the states of the cells were extracted as PLS scores. By using scores 1 and 2 that were used for extracting activated cells and naive cells, the training data were shown in the form of a kernel density estimation distribution. Thereafter, the test data (represented by dots) were superimposed on the training data (FIG. 37). By adopting the supervised machine learning method to combine it, it has become possible to classify activated cells and naive cells more accurately.

### (Inference of States of Jurkat Cells Using Irradiation Method According to Present Embodiment and Machine Learning)

It was examined whether same cell types in different states can be discriminated by using spectra obtained by performing the irradiation method according to the present embodiment. Jurkat cells (the human T cell line) were stimulated with magnetic beads bonded with CD3 and CD28 antibodies. It was forecasted that, in the stimulation process, a cell is activated via various kinds of states. Thus, after application of stimulation to the cells, sampling of the cells was performed at timing when respective periods of time, specifically, 0 hour, 0.5 hours, 3 hours, 6 hours, and 24 hours, have elapsed (FIG. 38). With respect to each of the sampled cells, the Raman spectrum was measured in a circular region having a diameter of 10 km.

Next, activation states of the cells were discriminated by using LDA. By using all data of the cells as training data, classification axes were created. Thereafter, as test data, visual classification was shown by using distribution of cells relating to the scores 1 and 2 extracted by LDA (FIG. 39). The cells used in the training data are shown in the form of kernel density estimation distribution, and unknown cells used in the test data were plotted on lines. It was possible to classify cells in different activation stages.

### (Measurement of Plural Cells)

Plural cells were measured at the same time by using the irradiation method according to the present embodiment.

Raman measurement of living cells using the present irradiation method was tested by using Jurkat cells. The region of measurement was set to be wide to make the region in such a manner that plural cells exist in the region, and the Raman spectra in a region having a diameter of 40 μm were measured. In measurement, with respect to each of cells existing in a circular region having a diameter of 40 μm, a spectrum in a circular region having a diameter of 10 μm was measured ((a) in FIG. 40). Further, regarding the cells existing in the circular region having the diameter of 40 μm, spectrum measurement was performed in such a manner that all the cells were measured collectively ((b) in FIG. 40).

Result thereof is shown in FIG. 41. With respect to any of the cases of measurement performed by using any of the methods, it was shown that there are characteristic peaks of nucleic acid (685, 748, 783 cm⁻¹), protein (1003, 1450 cm⁻¹), lipid (1125, 1580, 1654 cm⁻¹), and so on in the fingerprint region of 600-1800 cm⁻¹. Thus, in addition to the case wherein each of cells is measured individually, it was possible to measure cells existing in a designated region collectively.

### (Measurement of Plural Cells)

Plural cells were measured at the same time by using the irradiation method according to the present embodiment.

Raman measurement of living cells using the present irradiation method was tested by using human pancreas cancer cell line (Capan-1) cells and hMSCs. Capan-1 cells and hMSCs were cultured (both of them were put into the adhered states), respectively, and Raman spectra were measured in 10 spots in a region having a diameter of 80 μm. Regarding spectrum measurement, it was performed in such a manner that all the cells existing in the circular region having the diameter of 80 μm were measured collectively (FIG. 42).

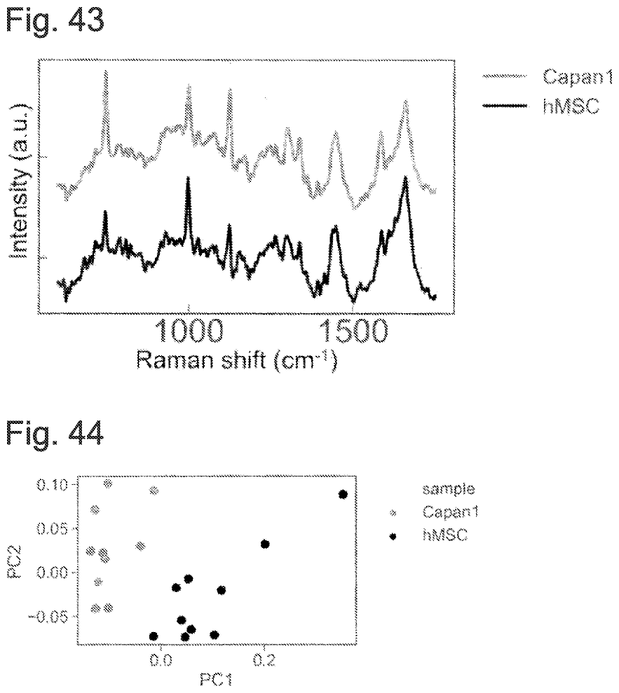

Average spectrum data of respective cells are shown in FIG. 43. Even if plural cells are measured collectively, it is possible to classify Capan-1 cells and hMSCs.

For testing discrimination of respective cell types, spectra obtained as multivariate data for principal component analysis (PCA), that is an unsupervised machine learning method, were used. The PCA extracted first two principal components (PCs) that contribute to dispersion of all cells. Contribution of the PCs were 58.98% and 11.68%. With respect to PC1 and PC2, the Capan-1 cells and the hMSCs showed different cell distribution densities.

Accordingly, it was confirmed that classification of cell types can be achieved, even if cells existing in a designated region are measured collectively.

### (Measurement of Plural Cells)

Plural cells were measured at the same time by using the irradiation method according to the present embodiment.

It was examined whether same cell types in different states can be discriminated. Jurkat cells (the human T cell line) were stimulated to obtain activated cells. Further, the cells were stimulated with PHA (phytohemagglutinin that is known as a T-cell activation factor) (1 μg/ml).

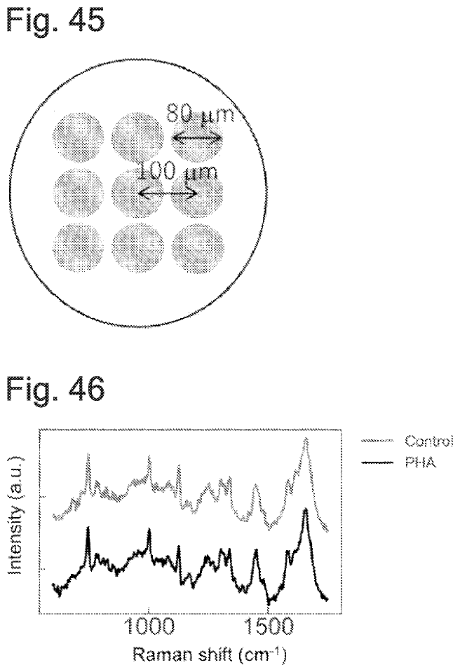

The cells, which were activated as explained above, and naive cells were seeded in separate wells, respectively; wherein the number of the activated cell and the number of the naive cells was 1.75×10⁷/ml. Regarding spectrum measurement, all cells existing in a region having a diameter of 80 μm were measured collectively by performing the present irradiation method. With respect to each cell, Raman spectra were obtained in a total of 18 regions (nine regions per one well) (FIG. 45).

Average spectrum data of respective cells are shown in FIG. 46. Even if plural cells are measured collectively, it is possible to classify activated cells and naive cells.

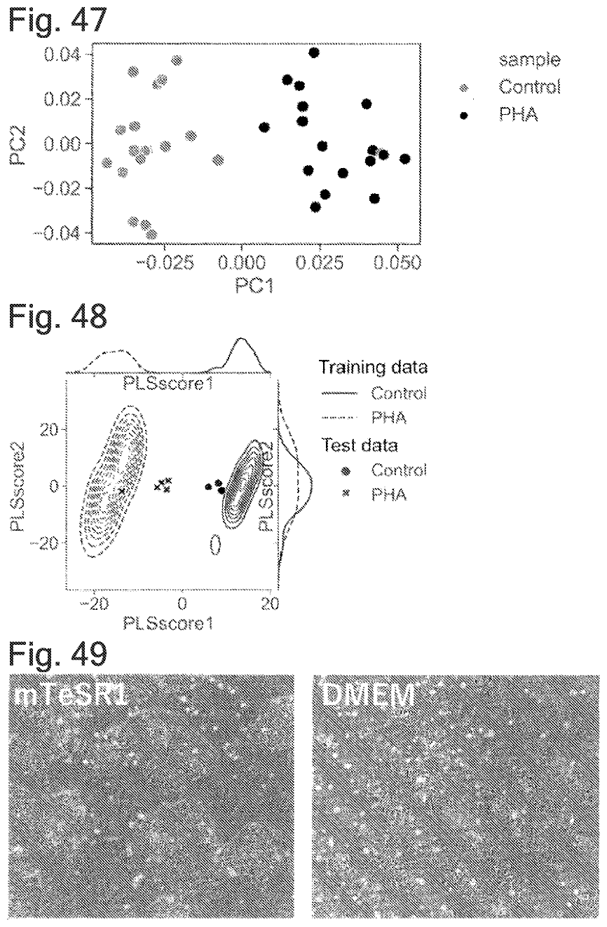

For testing discrimination of the activated cells and the naive cells, spectra obtained as multivariate data for principal component analysis (PCA), that is an unsupervised machine learning method, were used. The PCA extracted first two principal components (PCs) that contribute to dispersion of all cells. Contribution of the PCs were 10.21% and 4.16%. In relation to PC1 and PC2, the activated cells and the naive cells showed different cell distribution densities (FIG. 47).

Next, spectrum data were applied to supervised machine learning to attempt classification of cells. Partial least squares-discriminant analysis (PLS-DA) was used. For supervised machine learning, all data of the activated cells and the naive cells were randomly divided to training data accounting for 80% and test data accounting for 20%.

Two kinds of features that contribute to classification of states of the cells were extracted as PLS scores. By using scores 1 and 2 that were used for extracting activated cells and naive cells, the training data were shown in the form of a kernel density estimation distribution. Thereafter, the test data (represented by dots) were superimposed on the training data (FIG. 48).

Accordingly, it was confirmed that classification of activated cells and naive cells can also be achieved by measuring cells existing in a designated region collectively.

### (Measurement of Plural Cells)

Plural cells were measured at the same time by using the irradiation method according to the present embodiment.

For normally maintaining hiPSCs (the 201B7 line) and allowing subculture thereof in a feeder-cell-free state, a mTeSR1 medium (modified Tenneille Serum Replacer 1, which is available from STEM CELL) is used often. In such a case, hiPSCs form a rounded colony (FIG. 49). On the other hand, in the case that cultivation is performed by using a DMEM (Dulbecco's modified Eagle's medium) which is generally used for differentiated cells, hiPSCs cannot maintain the form of a normal colony, and exhibits an abnormal form such as a form wherein the outer periphery of the colony is made flat and so on (FIG. 49).

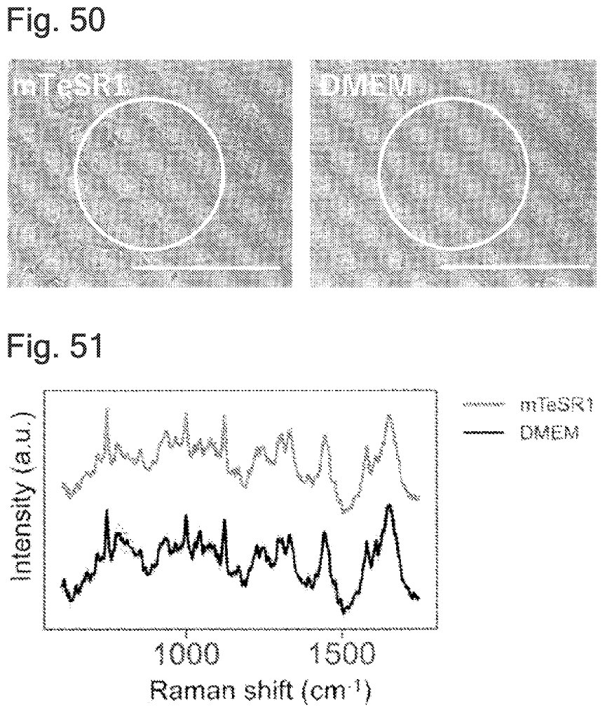

Raman spectrum data were obtained in each of a hiPSC colony having a normal form (cultured in a mTeSR1 medium) and a hiPSC colony having an abnormal form (cultured in a DMEM medium) by performing the present irradiation method, wherein the above colonies were those obtained by performing two types of culture method such as those explained above. Regarding spectrum measurement, all cells existing in a region having a diameter of 80 μm were measured collectively (FIG. 50). With respect to each cell, Raman spectra were obtained in a total of 20 regions (FIG. 51).

Spectrum data were applied to supervised machine learning to attempt classification of normal cells and abnormal cells. Partial least squares-discriminant analysis (PLS-DA) was used. For supervised machine learning, all data of the normal cells and the abnormal cells were randomly divided to training data accounting for 80% and test data accounting for 20%.

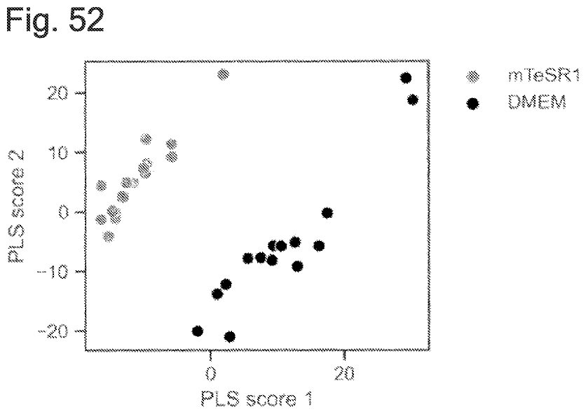

Two kinds of features that contribute to classification of states of the cells were extracted as PLS scores. By using scores 1 and 2 that were used for extracting normal cells and abnormal cells, the training data were shown in the forms of a dot plot (FIG. 52).

Accordingly, it was confirmed that classification of normal hiPSCs and abnormal hiPSCs can also be achieved by measuring cells existing in a designated region collectively.

According to the Raman spectroscopy system, the spectrum data obtaining method, and the cell classification method according the above-explained embodiments, laser irradiation can be performed more evenly, while reducing damaging of cells.

In the above description, embodiments of the present invention have been explained, and, in this regard, it is needless to state that the present invention is not limited to any of the above-explained embodiments, and can be implemented in any of various different modes within the scope of the technical idea of the present invention.

Further, the scope of the present invention is not limited to that of any of the illustrated embodiments described and shown in the figures, and it comprises all embodiments which provide effect that is equal to that the present invention aims to provide. Further, the scope of the present invention is not limited to that of any of combinations of characteristics of the invention defined by respective claims, and it can be defined by any desired combinations of specific characteristics in all of disclosed respective characteristics.

### REFERENCE SIGNS LIST

1 Raman spectroscopy system
12 DPSS laser light source
14 Spectrometer
16 CCD detector (Cooled CCD camera)
18 Inverted microscope
20 Raman optical system
30 Biaxial Galvano mirror
32 Laser optical fiber
34 Optical fiber
38 Shutter controller
40 Stage
42 Stage controller
44 Joystick
46 Epi-illumination controller
48 Epi-illumination device
50 Computer device
52 Keyboard
54 Mouse
56 Monitor device
202 Lens
204 ND filter
205 Shutter
206 Band-pass filter
208 Flat mirror
210 Dichroic mirror
212 High-pass filter
214 Lens
215 Shutter

1-3. (canceled)

4. A cell classification method comprising steps of:
receiving Raman scattered light that is outputted from a cell as a result that laser light is emitted in an approximately circular manner and in a turning manner to an approximately circular region where the cell exists;
detecting a Raman spectrum corresponding to the received Raman scattered light; and
classifying the cell based on spectrum data obtained from the detected Raman spectrum.

5. The cell classification method as recited in claim 4, wherein the step of classifying the cell comprises comparing the obtained spectrum data with standard spectrum data.

6. The cell classification method as recited in claim 4, wherein the step of classifying the cell is a step of classifying the kind of the cell.

7. The cell classification method as recited in claim 4, wherein the step of classifying the cell is a step of classifying cells in different states.

8. The cell classification method as recited in claim 4, wherein the step of classifying the cell is performed by using machine learning.

9. (canceled)

10. (canceled)

11. The cell classification method as recited in in claim 4, wherein, in the step of receiving Raman scattered light, the laser light is emitted in a turning manner to an approximately circular region in an individual cell.

12. The cell classification method as recited in in claim 4, wherein, in the step of receiving Raman scattered light, the laser light is emitted in a turning manner to an approximately circular region where plural cells exist.

13. The cell classification method as recited in in claim 4, wherein the cell is in an approximately spherical shape.

14. The cell classification method as recited in in claim 4, wherein the approximately circular region covers a nucleus and a cytoplasm of the cell.
